(19) United States
(12) Patent Application Publication
(10) Pub. No.: US 2022/0079760 A1
(43) Pub. Date: Mar. 17, 2022

(54) Annuloplasty Implant System and Associated Methods

(71) Applicant: Bluesail New Valve Technology Asia Ltd., Kowloon (HK)

(72) Inventors: Eugene Serina, Fremont, CA (US); Minh Nguyen, Midway City, CA (US); Sherrie Yang, Redondo Beach, CA (US); Yen Thai, Midway City, CA (US)

(73) Assignee: Bluesail New Valve Technology Asia Ltd., Kowloon (HK)

(21) Appl. No.: 17/475,086

(22) Filed: Sep. 14, 2021

Related U.S. Application Data
(60) Provisional application No. 63/077,843, filed on Sep. 14, 2020.

(51) Int. Cl. A61F 2/24

(52) U.S. Cl. CPC A61F 2/2448; A61F 2220/0008; A61F 2220/0016; A61F 2210/0014; A61F 2230/0095

(57) ABSTRACT

Annuloplasty implant systems and methods of deployment within a catheter-based procedure are provided herein. Such systems can utilize annuloplasty rings defined by a scaffold of braided wire that is radially expandable and axially collapsible or by annuloplasty rings defined by multiple interconnected concentric loops. The rings can be formed of a memory alloy and expand to an implantation configuration when advanced from the catheter to be secured against the valve annulus with the anchors. Deployment systems can include delivery catheters that deploy multiple anchors about a valve annulus, and advance the annuloplasty ring over multiple torque wires to securely couple the ring with the anchors forming the annuloplasty ring thereby reforming the valve annulus.

### CROSS-REFERENCE TO RELATED APPLICATIONS

The present application claims the benefit of priority to U.S. Provisional Application No. 63/077,843 filed Sep. 14, 2020, the contents of which is incorporated herein by reference in its entirety for all purposes.

The present application is generally related to co-pending and co-owned U.S. patent application Ser. No. 17/475,089 filed Sep. 14, 2021, the contents of which is incorporated herein by reference in its entirety for all purposes.

### BACKGROUND

Treatments for heart valve deficiencies, in particular mitral valve regurgitation, are widely varied. Mitral valve regurgitation is a condition that occurs when the mitral valve annulus is dilated or misshapen such that there is insufficient coaptation between the posterior mitral leaflet (PML) and the anterior mitral leaflet (AML), which allows blood to flow backward from the left ventricle (LV) into the left atrium of the heart (see heart anatomy in FIG. 1). Over time, this deficiency worsens and can lead to congestive heart failure, atrial fibrillation, pulmonary hypertension and ultimately death. Among the earliest approaches to mitral valve repair is the prosthetic annuloplasty ring developed in 1968. The prosthetic aimed to reform the proper shape of the valve annulus to provide proper leaflet coaptation so that normal valve function was restored. As compared to earlier approaches, the prosthetic annuloplasty ring to remodel the shape of the valve annulus has provided consistent and reliably positive patient outcomes and long-lasting results. One major drawback of this early approach, however, is that the annuloplasty ring is manually sutured into place around the valve annulus so that the implantation required an open-heart surgical procedure, which present considerable risks and challenges, particularly for patients already in poor health. In recent decades, a number of catheter-based approaches have been developed that attempt to similarly remodel the shape of the valve annulus while avoiding the risks associated with an open-heart surgical procedure. These catheter-based approaches include a variety of approaches, including cinching implants, leaflet clips, as well as sutures and splints that span across a heart cavity. However, few if any approaches thus far have provided the consistency and reliability in implantation and patient outcomes as the original prosthetic annuloplasty ring approach noted above. In addition, as with many catheter based procedures, precise placement and implantation is more challenging due to the enclosed environment and limited visualization. Accordingly, these catheter-based procedures can be tedious and time-consuming, with the outcome of the procedure often heavily reliant on the skill of the physician. While more recent developments have sought to replicate the advantages of a prosthetic annuloplasty ring within a catheter-based approach, as of yet, these approaches have so far failed to replicate the success of a convention surgically implanted annuloplasty ring, due largely to the complexities in anchoring and securing the annuloplasty ring. Thus, there is need for a catheter-based approach that allows for improved ease and consistency in implantation. There is further need for improvement in prosthetic annuloplasty ring technologies.

### BRIEF SUMMARY

The present disclosure relates to annuloplasty implant systems, associated delivery catheters and components as well as methods of deployment. While the systems and methods are described in regard to treatment of the mitral valve, it is appreciated that these concepts can be applicable to any heart valve and any implant anchored within a body lumen.

In one aspect, the invention pertains to an improved annuloplasty ring. In some embodiments, the annuloplasty ring can be a scaffold formed by one or more wires that are braided circumferentially about a central opening extending along a longitudinal axis of the scaffold, wherein the scaffold is expandable from a delivery configuration to a deployed configuration. In the delivery configuration, the scaffold has first axial dimension and a first diameter about the central opening, the first axial dimension being greater than the first diameter and in the deployed configuration, the scaffold has a second axial dimension and a second diameter about the central opening, the second diameter being greater than the second axial dimension. In other embodiments, the annuloplasty ring can be defined by multiple concentric rings interconnected by multiple struts so that the multiple rings are axially separable or movable, at least partly. The ring can be formed of a shape memory alloy, such as Nitinol, that is heat set to an implantation configuration that corresponds to desired characteristics of a native heart valve, and the multiple rings are deformable into a contracted configuration for delivery through a catheter and return to the implantation configuration when released from the catheter during implantation. The ring can include eyelets distributed circumferentially about the ring for interfacing with multiple anchors or anchor wires. Typically, the concentric rings have substantially similar 2-dimensional shapes that corresponds to a desired shape of the valve annulus. In some embodiments, the multiple concentric rings have differing 3-dimensional shapes so that, when combined, the annuloplasty ring has a radial strength and flexibility corresponding to the desired characteristic of the native valve annulus. In one aspect, the rings are customized to provide a shape, strength and flexibility for the valve annulus of a particular patient. In some embodiments, the ring includes collars at each eyelet that facilitate sliding over the torque tubes or wires. The collars can further include a coupling feature or mechanism, such as any of those described further below, for securing the ring to the anchors. In some embodiments, each collar include one or more inwardly deflectable tabs configured to engage with a lock mechanism of a corresponding anchor. In some embodiments, the lock mechanism on the anchors includes one or more hypotubes, each having a proximal tapered portion to facilitate passage of the tabbed collar thereon and a flat distal facing surface that abuts against the tabs of the collar to attach the implant. The lock mechanism can include a series of such hypotubes along the shaft so as to be adjustable. It is appreciated that any of these ring designs could be utilized in accordance with the various features and embodiments described herein.

In another aspect, the invention pertains to an annuloplasty implant system. The system can include multiple anchors, each anchor including a shaft extending between proximal and distal ends, a distal penetrating anchor disposed at the distal end, a ring locking feature disposed along the shaft and configured for coupling with the annuloplasty ring, and a couple-release mechanism disposed along the shaft and configured for coupling and releasing a torque wire. The system further includes an annuloplasty ring having an implantation configuration of a set-shape corresponding to desired characteristics of a valve annulus. The annuloplasty ring can include multiple eyelets, each eyelet sized and configured to receive a respective anchor shaft and securely couple thereto via the lock feature. In some embodiments, the ring locking feature and torque wire coupled-release mechanism are configured so that actuation of the ring locking mechanism to secure the shaft with the annuloplasty ring effects actuation of the couple-release mechanism to decouple shaft from the torque wire. In some embodiments, the ring locking mechanism includes an inwardly biased ridge on an inside of a collar attached to the ring that engages a shoulder or flange on the shaft of the anchor. The torque wire couple-release mechanism can include interlocking protruding features on the shaft and distal end of the torque wire, that when engaged couple the anchor and torque wire and when disengaged, decouple the torque wire. In some embodiments, the anchor release mechanism includes a longitudinally translatable core wire extending through the torque wire that, when present, forces a locking component outward to engage a slot in an outer tube of the anchor. Retraction of the core wire allows the locking component to resiliently deflect inward, thereby disengaging from the slot of the outer tube to detach the torque tube from the anchor. In other embodiments, the release mechanism can include a rotating cam lock that is rotatable between a locked position in an outer sleeve and an unlocked position in which the cam lock can be withdrawn from the sleeve. In some embodiments, the locking mechanism can include a hook coupling that releasably attaches to the ring at eyelets or collars and extends through a hole in the anchor when the ring is advanced, thereby locking the ring to the anchor. In other embodiments, the ring locking mechanism can include a ball-detent coupling in which a spring-loaded ball extends from a collar of the ring and through a hole or detent in the anchor, thereby locking the ring to the anchor.

In yet another aspect, the invention pertains to a method of implanting an implant system for reshaping a valve annulus of a heart of a patient. The method can include steps of: implanting multiple anchors within tissue surrounding the valve annulus, each anchor including a distal tissue penetrating anchor and a proximal shaft having a lock mechanism and a couple-release mechanism; advancing an annuloplasty ring over the proximal shafts of multiple anchors until disposed substantially against the valve annulus; locking the annuloplasty ring via the lock mechanism by further advancing the annuloplasty ring distal of the lock mechanism; and releasing the couple-release mechanism, thereby releasing the torque wires from multiple anchors while the annuloplasty ring remains secured against the valve annulus by the lock mechanism. Implanting the anchors can include actuating multiple torque wires coupled with the multiple anchors via the couple-release mechanisms. The anchors and the annuloplasty rings are delivered intravascularly from one or more catheters. In some embodiments, the anchors are delivered from a first catheter through an access sheath and the annuloplasty ring is delivered from a second catheter through the access sheath. In some embodiments, the lock mechanism and the couple-release mechanism are configured so that actuation of the lock mechanism by advancement of the annuloplasty ring effects release of the couple-release mechanism, thereby releasing the torque wires. In some embodiments, the method further includes assessing valve function by visualization techniques after initially advancing the annuloplasty ring substantially against the valve annulus and before locking the annuloplasty ring.

In another aspect, the invention pertains to an implant delivery system for delivering an implant. The system can include a delivery catheter configured to extend from outside the patient to within the patient; an implant disposed within a distal portion of the delivery catheter, the implant having at least one or more wire loops; and one or more pusher members extendable along the length of the catheter. In some embodiments, each of the one or more pusher members includes an implant holding-release mechanism that is actuatable between a locked position and a released position. In some embodiments, the implant holding-release mechanism comprises a spring-loaded sleeve having an inner hypotube sleeve and an outer hypotube sleeve that are axially movable relative each other by a pull wire and biased to a locked position by a spring. In the locked position, the spring maintains the inner hypotube sleeve axially extended so that the wire is constrained between the inner and outer hypotube sleeve. In the released position, the inner hypotube sleeve is retracted thereby releasing the wire loop. The catheter further includes a proximal handle of the catheter that controls advancement of the one or more pusher members and retraction of the pull wire of the one or more pusher members during implant delivery. In some embodiments, the inner and outer hypotube sleeves have wedge surfaces that interface in the released position so as to push the wire loop of the implant outward from the inner and outer hypotubes. In some embodiments, the catheter includes a plurality of pusher members that engage differing portions of the one or more wire loops and that can be advanced concurrently and/or individually. In the example described herein, the implant includes an annuloplasty ring comprised of multiple concentric rings or braided construct having eyelets or collars that are pushed by multiple pusher members onto multiple anchors deployed around an annulus of a heart valve.

### BRIEF DESCRIPTION OF THE DRAWINGS

FIG. 1A shows a cross-sectional side view of an implanted annuloplasty implant system, in accordance with some embodiments of the invention.

FIGS. 1B-1C show the anatomy of the mitral valve.

FIGS. 2A-2D show a conventional prosthetic annuloplasty ring implanted in an open-heart surgical procedure.

FIG. 3 shows an anchor delivery catheter in accordance with some embodiments.

FIG. 4A shows a distal anchor delivery portion of the anchor delivery catheter in accordance with some embodiments.

FIG. 4B shows a proximal control handle of the anchor delivery catheter in accordance with some embodiments.

FIGS. 5A-5C show several views of a screw anchor in accordance with some embodiments.

FIGS. 6A-6B show a torque wire and anchor coupled and decoupled by a torque wire couple-release mechanism, respectively, in accordance with some embodiments.

FIGS. 7A-7D show cross-sectional views of the torque-wire couple-release mechanism of the embodiment of FIGS. 6A-8B.

FIGS. 8 and 9A-9B show an alternative coupling-release mechanism having a rotatable cam lock in accordance with some embodiments.

FIGS. 10A-10C show an adjustable ring locking feature for securing the ring to the anchors in accordance with some embodiments.

FIGS. 11A-11B show alternative ring locking features. FIG. 11A shows a ring locking feature having a hook coupling for securing the ring to the anchors in accordance with some embodiments. FIG. 11B shows a ring locking feature having a ball-detent coupling for securing the ring to the anchors in accordance with some embodiments.

FIGS. 12A-12D show several views of an annuloplasty ring design in accordance with some embodiments.

FIGS. 13A-14B show an adjustable annuloplasty ring design in accordance with some embodiments.

FIG. 15A shows an exemplary annuloplasty ring design configured to slide on multiple cables in accordance with some embodiments.

FIGS. 15B and 15C show the annuloplasty ring of FIG. 15A in a delivery configuration and a deployed implantation configuration, respectively, in accordance with some embodiments.

FIG. 15D shows an exemplary annuloplasty implant system implanted on a model of a mitral valve annulus in accordance with some embodiments.

FIGS. 16A-16B show an exemplary annuloplasty ring with polymer sutures wrapped on select wires to promote tissue ingrowth, in accordance with some embodiments.

FIGS. 17A-17D show an exemplary annuloplasty ring having a D-shape and curved saddle shape to better conform with a natural shape of the annulus, in accordance with some embodiments.

FIGS. 18A-18B show views of an annuloplasty ring being deployed from an annuloplasty ring delivery catheter in accordance with some embodiments.

FIGS. 19A-19C show several views of an annuloplasty ring delivery catheter in accordance with some embodiments.

FIG. 20 shows an articulable access sheath that can be advanced intravascularly to an atrium of the heart, such as in a transfemoral approach, to provide access for the respective delivery catheters of the anchors and annuloplasty ring in accordance with some embodiments.

FIG. 21 shows the access sheath advanced and penetrating through the septal wall and into the left atrium to provide access to mitral valve in the left atrium.

FIGS. 22A-22H show sequential views of delivery and implantation of the annuloplasty implant system in accordance with some embodiments.

FIG. 23 shows an exemplary ring coupling/release mechanism in accordance with some embodiments.

FIGS. 24 and 25 show locked and unlocked positions, respectively, of the exemplary ring coupling/release mechanism in accordance with some embodiments.

### DESCRIPTION OF THE INVENTION

The present invention pertains to an implants system and associated delivery catheters and methods of delivery that seek to provide similar reliability and consistency in patient outcomes as a conventional prosthetic annuloplasty ring implanted in an open-heart surgical procedure. Advantageously, the invention allows for a similar approach but within a minimally invasive catheter-based approach. In one aspect, the system separates deployment of the anchors from deployment of the annuloplasty ring, thereby allowing the physician greater focus on proper anchor placement and implantation before implantation of the annuloplasty ring. The invention further allows for improved ease of use and time efficiency by allowing the physician to implant multiple anchors simultaneously, while still allowing for independent anchor deployment as needed to ensure optimal placement of all anchors. In another aspect, the invention provides for an improved three-dimensional (3D) annuloplasty ring that allows for improved reformation of the valve annulus as compared to a conventional annuloplasty ring. While the system and methods described herein utilize this improved 3D annuloplasty ring, it is appreciated that the anchor deployment catheter and methods can be used with a variety of different types of annuloplasty rings, including two-dimensional (2D) annuloplasty rings. Further, it is appreciated that the improved 3D annuloplasty ring can be used with various other anchor deployment technologies and still provide the benefits of its improved design.

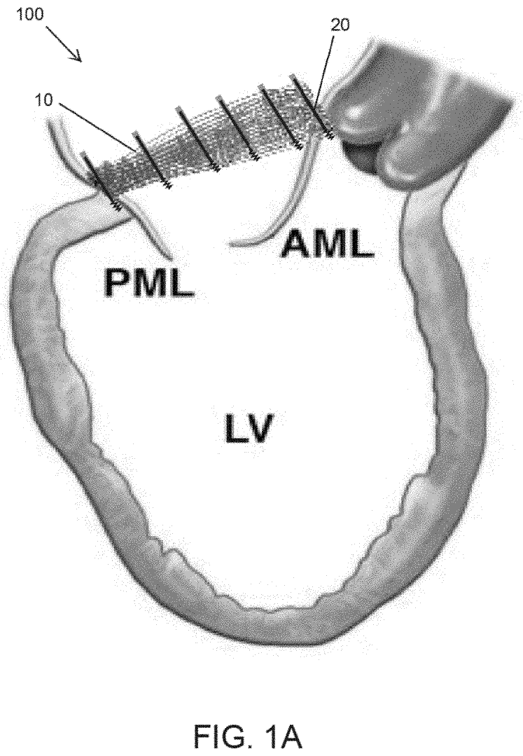

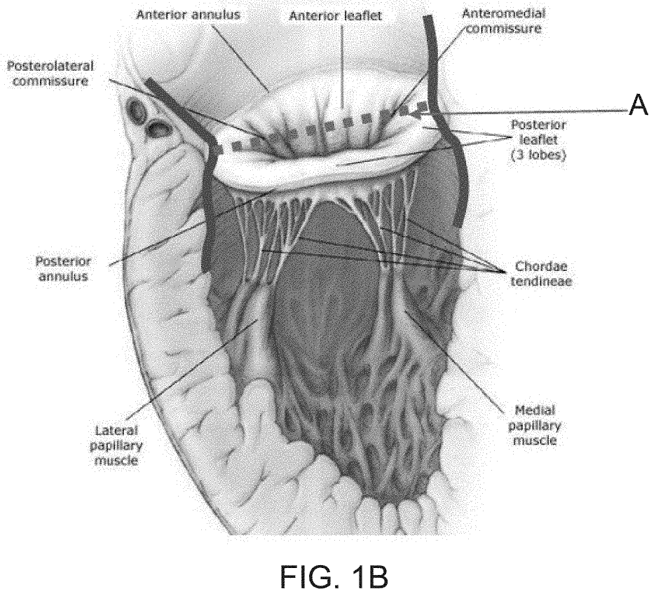

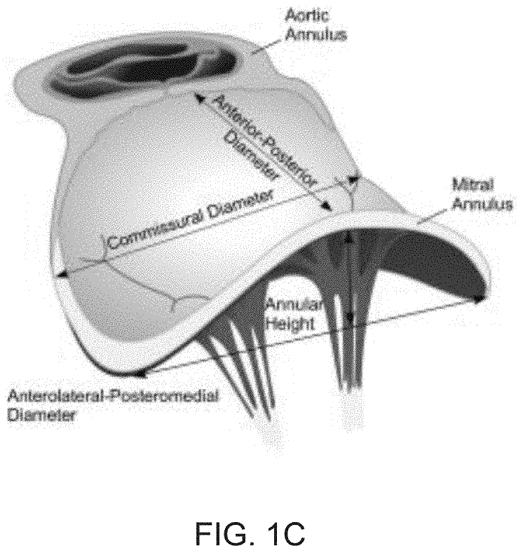

FIG. 1A shows a cross-sectional side view of an exemplary annuloplasty implant system 100 in accordance with some embodiments. The implant system includes multiple screw anchors 20 that are implanted in tissue surrounding the mitral valve annulus. The anchors are implanted at positions distributed evenly about the valve annulus. In some embodiments, the anchors are distributed unevenly, for example at location where more anchoring forces are needed due to the morphology of the valve. Typically, between 5-20 anchors are used, typically within a range of 6 to 12, preferably about 8 anchors, although any suitable number of anchors can be used. A 3D annuloplasty ring 10 is disposed adjacent the valve annulus and securely locked to the anchors by a ring locking mechanism, thereby reforming the shape of the valve annulus. The annuloplasty ring 10 can be specially configured to reform the 3D shape of the valve annulus to improve coaptation of the AML, and PML, leaflets and restore normal valve function. The means by which the implant system is delivered and implanted is described in detail below. FIGS. 1B and 1C shows the anatomy of the mitral valve and in particular the location of the annulus A relative the atrium above the annulus and the ventricle below the annulus. As can be seen in FIG. 1C, the natural shape of a healthy mitral valve annulus generally has a D-shaped two-dimensional shape and a three-dimensional shape that is saddle-shaped.

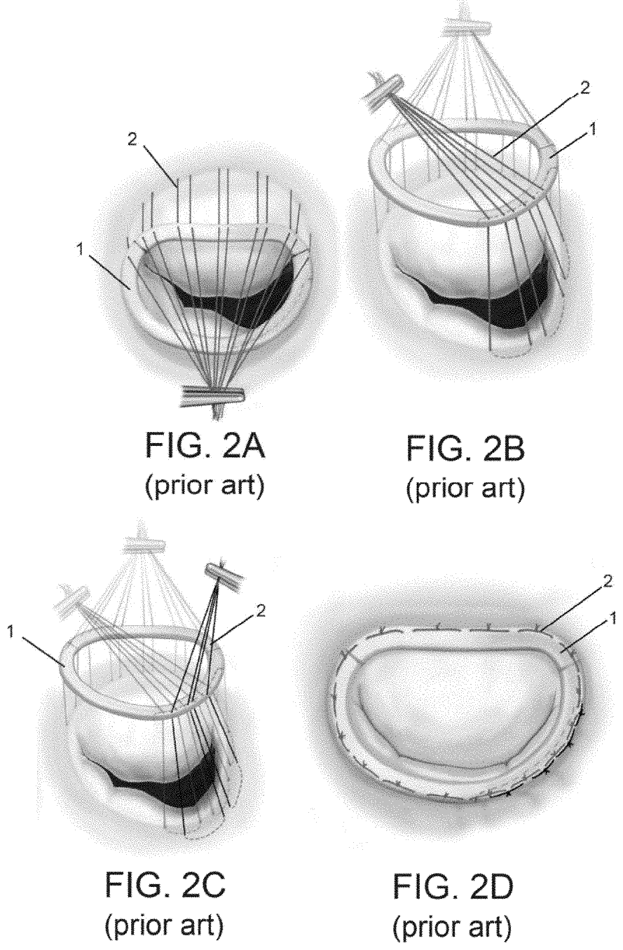

FIGS. 2A-2D show a conventional annuloplasty ring implantation in an open-heart surgical procedure. This conventional procedure is often considered the gold standard in surgical of mitral regurgitation repair and involves implantation of a semi-rigid annuloplasty ring 1 around the valve annulus. As shown in FIG. 2A, sutures 2 are implanted along the valve annulus, spaced precisely around the valve annulus. The sutures 2 are then sewn through the smaller sized annuloplasty ring 1, as shown in FIG. 2B. As shown, the spacing of the sutures is smaller on the ring. The ring is then pushed down upon the annulus, as shown in FIG. 2C, drawing the dilated valve annulus to the smaller diameter of the annuloplasty ring. The sutured are then tied off completing the repair, as shown in FIG. 2D. As noted above, this approach has provided reliably consistent results, yet suffers the considerable drawbacks associated with manually suturing tissues in an open-heart surgical procedure.

In one aspect, the annuloplasty implant system of FIG. 1A is designed to replicate the conventional annuloplasty ring surgical procedure, depicted in FIGS. 2A-2D, in order to provide similar consistency and reliability in patient outcomes. Advantageously, the concepts described herein allow this procedure to be performed in a catheter-based approach (e.g. a transfemoral catheter approach) that avoids the drawback and risks associated with an open-heart surgical procedure. In one aspect, the implantation method of the annuloplasty implant system described herein involves two main steps: (i) delivering and deploying multiple anchors with cables; and (ii) delivering an annuloplasty ring over the cables to secure with the anchors. Separating anchor deployment from ring deployment allows for greater design focus on improving ease and consistency in positioning and implanting the anchors around the valve annulus. In another aspect, this approach allows for use of an improved annuloplasty ring design having a 3D shape that remodels the valve annulus to a more anatomically correct shape and leads to better clinical performance. Conventional annuloplasty rings typically have a 2D shape (e.g. flat), which neglect the contours and morphology of the patient's natural valve annulus. Utilizing a 3D shape allows for an annuloplasty ring that can not only conform to the patient's morphology, but can also reform the overall shape and contours of the valve annulus to a desired 3D shape, rather than just reducing the diameter to a 2D shape. In some embodiments, this improved annuloplasty design can be customized specifically for a patient's anatomy to reform the valve annulus to the desired form.

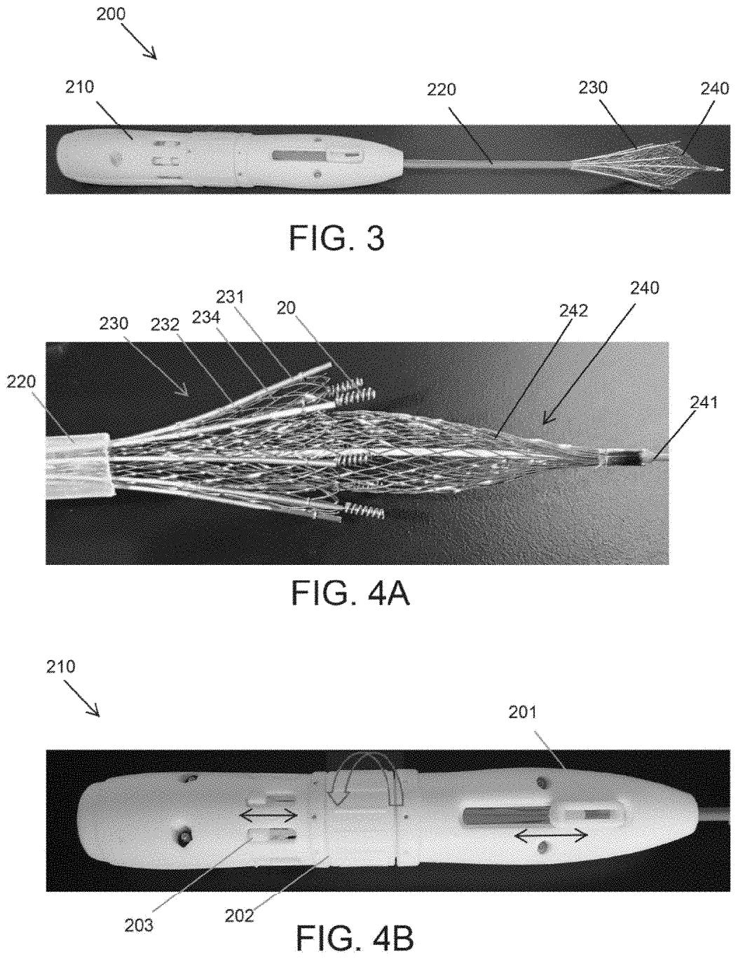

FIG. 3 shows an anchor delivery catheter 200 in accordance with some embodiments. Anchor delivery catheter 200 includes a proximal handle 210, an elongate flexible shaft 220, and an expandable anchor support 230 and expandable centering member 240 that are advanceable from the distal end. In some embodiments, the anchor support 230 and centering member 240 are each expandable frames, scaffolds or baskets, the anchor support 230 being an outer basket and the centering member 240 being an inner basket such that expansion of the inner basket expands the outer basket. In some embodiments, the centering member is a balloon, however, in this embodiment, the centering member is a scaffold or basket, which is advantageous as it allows blood to circulate while the centering member is expanded. In addition, the centering member is separable from the anchor support such that the centering member can be contracted while the anchor support remains expanded, which allows the valve to function while the anchors are adjusted and/or driven into the tissue. This also allows the physician to spend more time to accurately position and reliably deploy the anchors, as compared to systems where centering structures are integral with the anchor deployment mechanism.

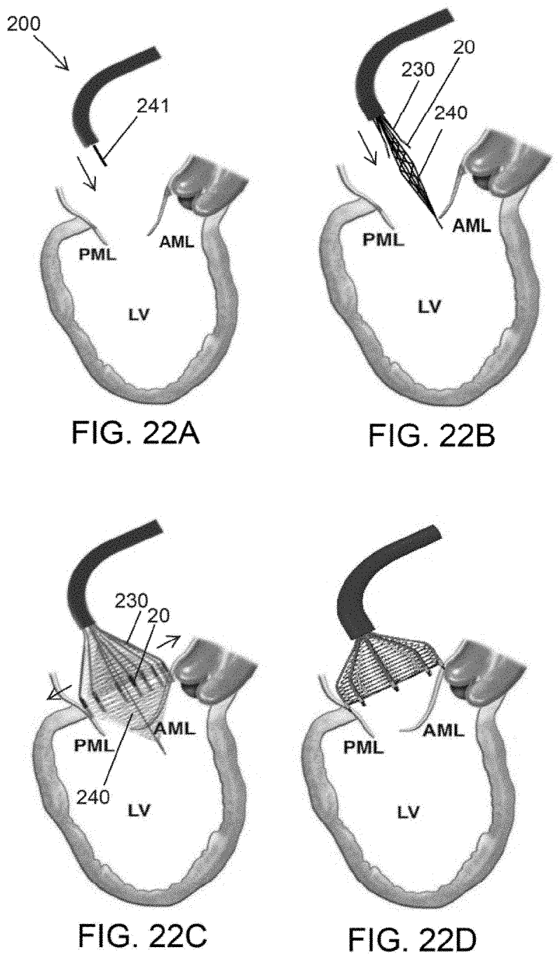

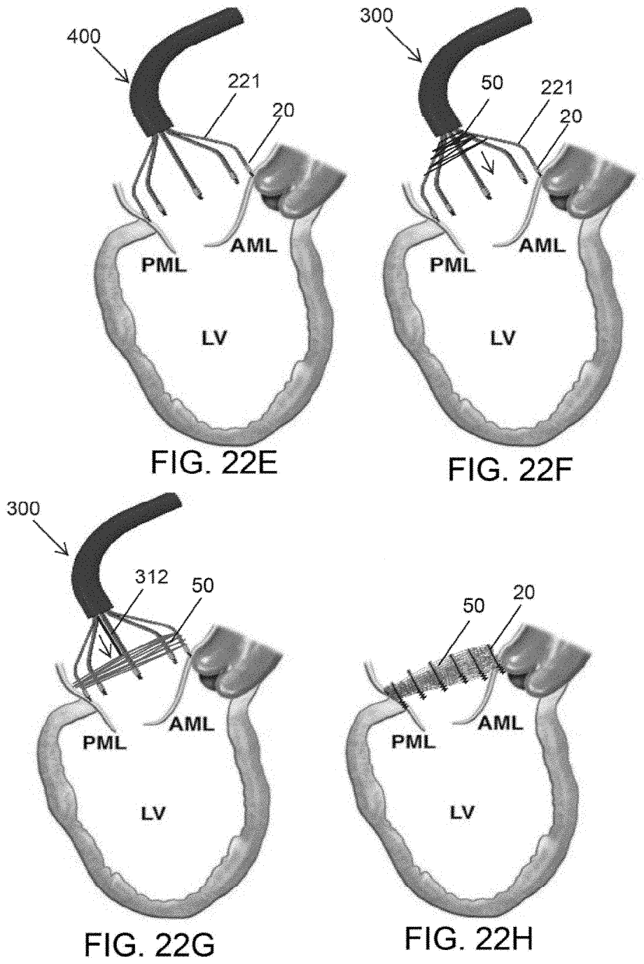

FIG. 4A shows a detail view of the distal portion of the anchor delivery catheter 200. The anchor support 230 includes support guides 231 with torque wires (not visible) therein. Multiple screw anchors 20 are releasably coupled to the distal ends of the torque wires and extend distally of the support guides 231. In some embodiments, the catheter includes between five and ten anchors, preferably about eight anchors, disposed radially about the anchor support. Torquing of the individual torque wires, by torque mechanisms that are disposed within the handle, drives each anchor 20 into the tissue after positioning of the anchors about the valve annulus. The support guides 231 are evenly spaced and may be interconnected by an expandable struts, mesh or frame 234 extending between the support guides. The distal portion of the support guides 231 splay outward so that the distal anchors are spaced apart from the centering member, which avoids interference between the anchors and centering basket during anchor delivery. The distal portion of the support guides 230 also include a spring portion 232, which allows the anchor support frame and anchors to be more conformable during delivery and allows for more uniform anchor and tissue interaction before deployment. The centering member 240 includes a central shaft 241 to which is attached an expandable mesh or basket 242 that when foreshortened expands laterally outward. For example, axial movement of the central shaft from the proximal handle expands and contracts the centering member 240 to facilitate centering during anchor delivery. As discussed in more detail in FIGS. 22A-22D, the anchor support 230 and centering member 240 are advanced from the distal end of catheter 200, the centering member is expanded, thereby centering the assembly within the valve annulus and also expanding the anchor support thereon to position the anchors about the valve annulus. Further advancement engages the anchors with the tissue surrounding the valve annulus, after which the centering member can be contracted and withdrawn to allow blood flow while the anchors are implanted into the tissue.

FIG. 4B shows a proximal control handle 210 of the anchor delivery catheter and includes control features for controlling delivery and deployment of the anchors. Centering switch 201 effects axial linear motion for opening and closing of the centering basket 240. Torque actuator 202 engages torque mechanisms that torque the individual torque wires for rotational deployment or removal of anchors. Rotation of torque actuator 202 in one direction (e.g. clockwise) effect clockwise rotation of engaged torque wires to screw anchors into tissue, while rotation of the torque actuator 202 in the opposite direction effects counter-clockwise rotation of engage torque wires to effect removal of anchors. This feature allows for simultaneous deployment of all screw anchors 20. Selector switches 203 allows the physician to select one or more individual anchors to apply torque for removing one or more anchors, after which the physician can adjust or reattempt deployment on an individual basis. As shown, moving the switch 203 in one direction engages the torque tube with the torque mechanism such that rotation of actuator 2 effects torquing of the respective torque wire, while moving the switch in the opposite direction disengages the torque wire from the torque mechanism such that the respective torque tube is not torqued when the actuator 2 is rotated. This feature allows a physician to select any, all or any combination of anchors for deployment. However, if the position of a single anchor is then determined to be suboptimal by visualization techniques, an individual anchor can be selected and removed, repositioned as needed, then subsequently redeployed into the tissue.

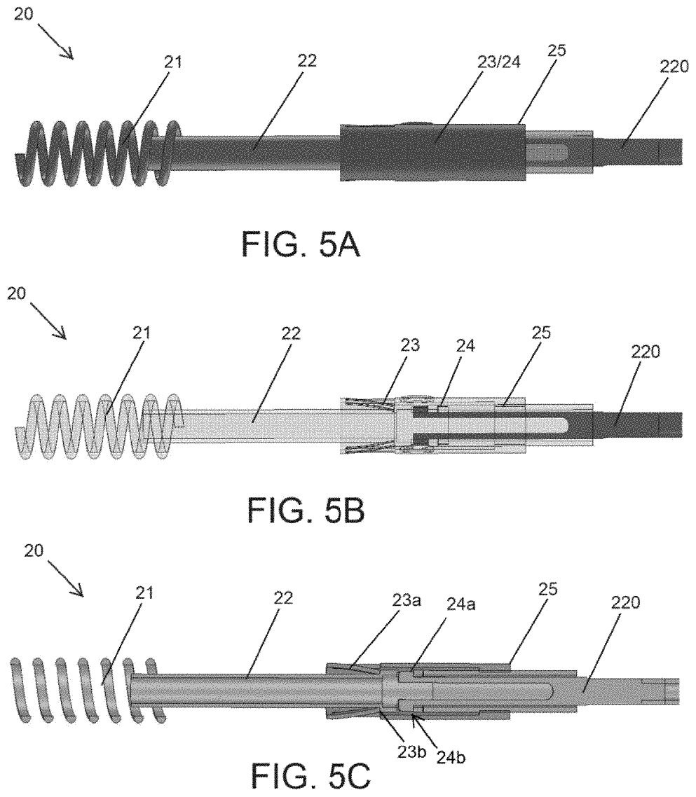

FIGS. 5A-5C show several views of screw anchors 20 in accordance with some embodiments. As described above, the anchors are analogous in function to the sutures in a conventional annuloplasty procedure. Each anchor 20 includes a distal penetrating tip 21 and a proximal shaft 22. In this embodiment, the distal tip is a helical screw that engages tissue and implants by rotation. Components of a locking mechanism 23, and a couple-release mechanism 24 are disposed on a proximal region of the shaft 22. The ring lock mechanism 23 secures a locking collar 25 attached to the annuloplasty ring (not shown) to the anchor shaft. The torque wire couple-release mechanism 24 couples the torque wire 220 to the proximal end of shaft 22 to facilitate driving of the screw anchor into tissue by torque of the torque wire and decouples the anchor from the torque wire when the ring is positioned and reformation of the valve annulus is determined to be sufficient.

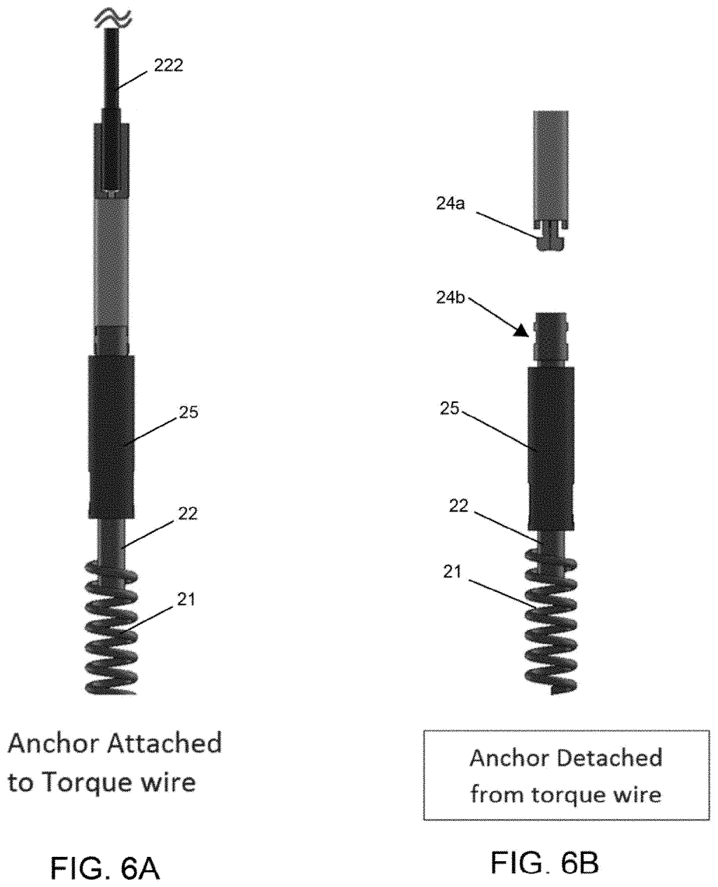

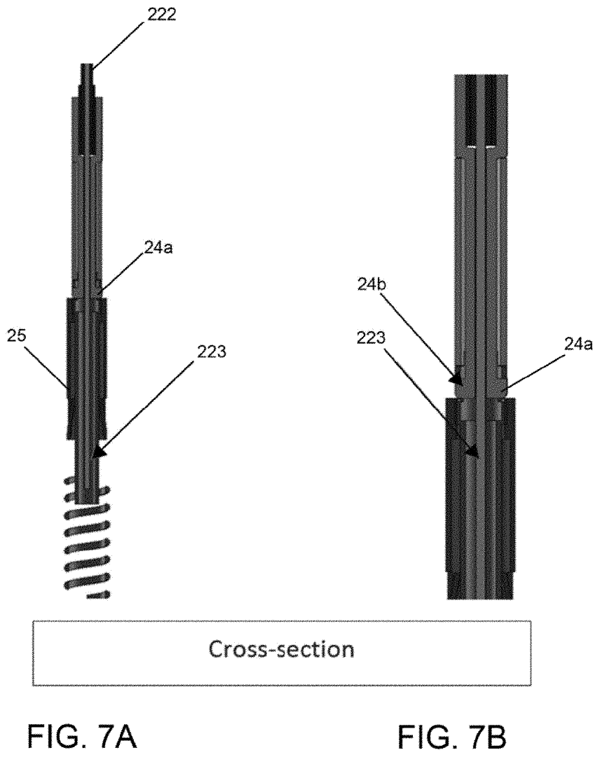

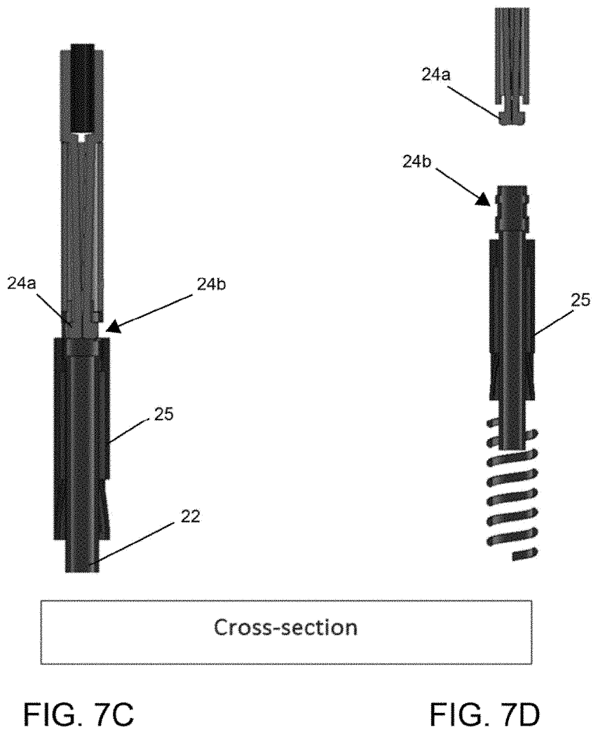

In the embodiment shown, the ring locking mechanism 23 includes a ridge 23a within the locking collar 25 that is inwardly biased in a proximal direction such that advancing the ring and locking collar 25 beyond a shoulder 23b on a proximal region of the anchor shaft 22, causes ridge 23a to deflects inwardly toward anchor shaft 22 and abut against the shoulder 23b, thereby locking the collar 25 and attached ring to the anchor. The couple-release mechanism 24 can includes a slot 24b at a proximal end of the anchor shaft 22 that receives a corresponding distal ridge 24a on inwardly biased members at a distal end of the torque wire so as to interlock and couple the torque wire with the anchor shaft. The operation of the torque wire couple-release mechanism 24 is further depicted in FIGS. 6A-6B and 7A-7D.

FIG. 6A shows the anchor shaft 22 attached to the torque wire 222 with locking collar 25 (ring not shown) locked to the anchor shaft. FIG. 6B shows the torque wire 222 detached from the anchor shaft 22, disengaged by the couple-release mechanism 24. As shown, the ridge 24a is disposed on inwardly biased members that deflect inwardly upon removal of an inner core wire 223 so that ridge 24a disengaged from slot 24b along the proximal end of anchor shaft 22. FIGS. 7A-7B show cross-sectional views of the assembly before and after release of the torque wire 222 after the locking collar 25 with ring (not shown) has been secured to the anchor. As shown in FIGS. 7A-7B, central core wire 223 extends through torque tube 222 forcing the inwardly biased members apart so that distal ridge 24a extends laterally outward into the slot 24b of the anchor shaft 22, thereby locking torque wire 222 to the anchor. As shown in FIG. 7C, when core wire 223 is removed, the inwardly biased members of locking component 24a recover to their stress free state so that the members are drawn inward and ridge 24a is removed from slot 24b, thereby disengaging from the anchor shaft 22 to allow withdrawal of torque wire 222, as shown in FIG. 7D.

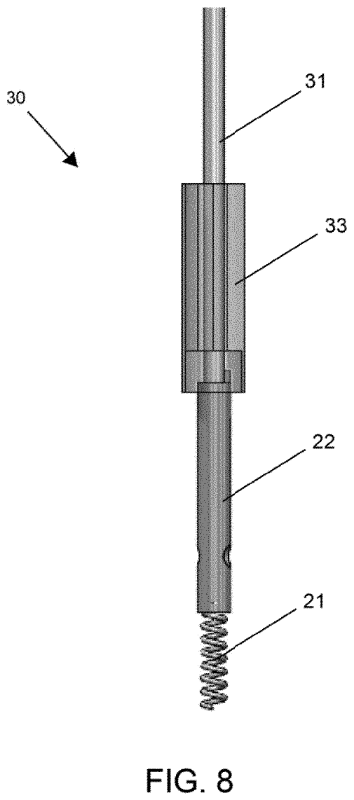

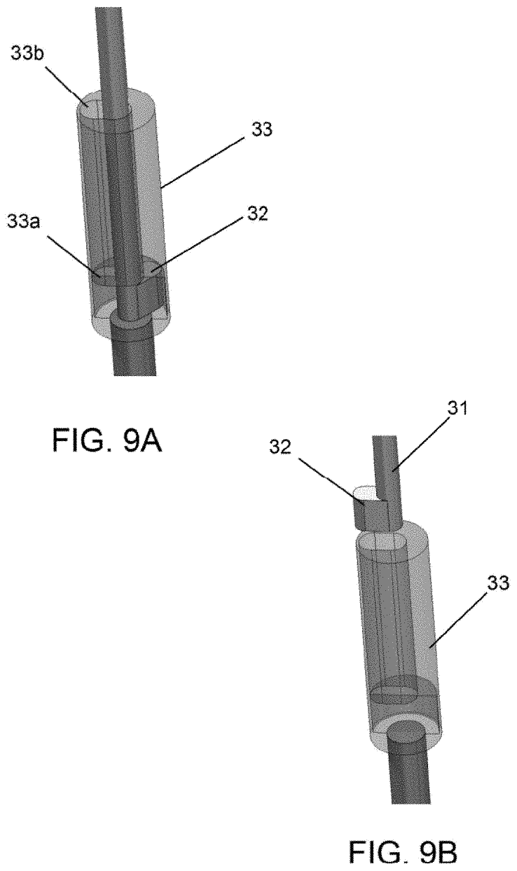

In another embodiment, the couple-release mechanism can include a rotating cam lock. As shown in the embodiments of FIGS. 8-11, the rotating cam lock 30 can include a cam lock 31 that interfaces with a locking sleeve 33 attached to the anchor shaft 22. As shown in the detail views of FIGS. 9A-9B, cam lock 31 includes a shaft and a distal cam 32 that can be positioned in a locked position (see FIG. 9A) during anchor delivery and deployment. As shown, the cam 32 is in a turned locked position within a corresponding shaped cavity 33a within the distal portion of the locking sleeve 33, which prevents the cam lock and attached torque tube from sliding out of the locking sleeve. After the annuloplasty ring is placed and secured to the anchors, the torque wires are released by twisting the cam lock 31. The cam lock 31 shaft can be rotated from their proximal end outside the patient, which rotates the cam 32 to align with a longitudinally extending slot 33b to allow cam 32 to be proximally retracted from the locking sleeve 33, thereby releasing the torque wires from the anchors.

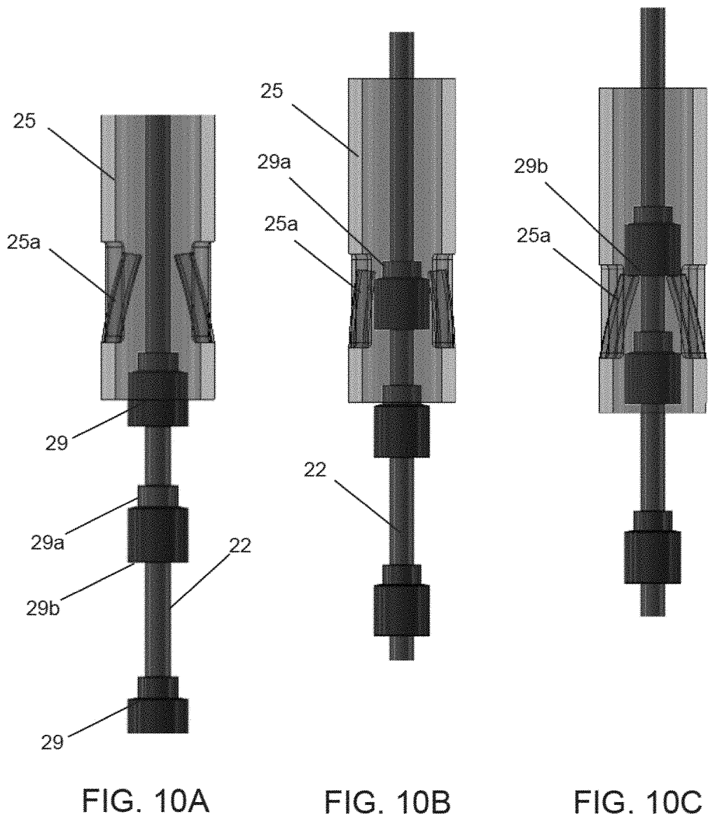

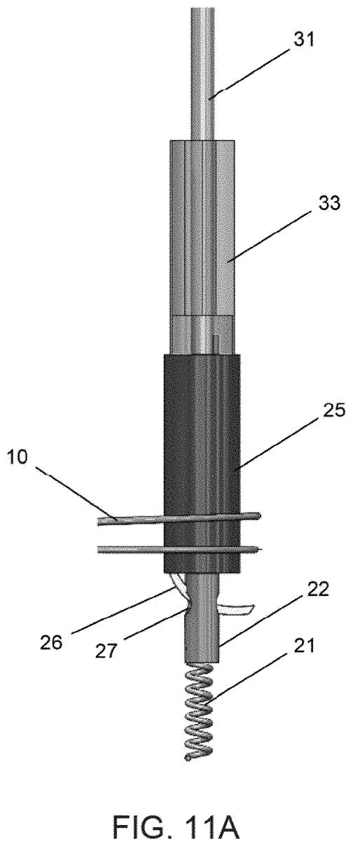

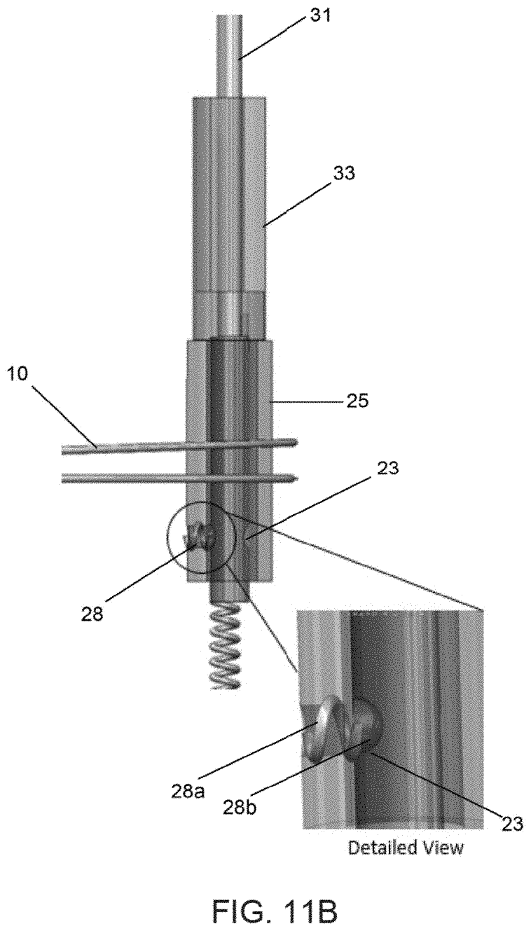

In another aspect, the ring locking mechanism can include a protruding element of a locking collar attached to the ring that interfaces with a hole or recess within the anchor body. Examples of such mechanisms are shown in the embodiments in FIGS. 10-11. In one embodiment, the ring coupling mechanism includes a hook coupling in which a hook or resiliently biased member on the annuloplasty ring or attached locking collar interface with a hole or recess on the anchor.

As shown in FIGS. 10A-10C, the anchor shaft 22 can include one or more hypotube features 29 that lock against one or more inwardly extending tabs 25a of the collars 25 inclined in the proximal direction. In this embodiment, the anchor includes a series of three hypotube features 29, which allows for adjustability, and the collar includes at least two inwardly extending tabs. As can be seen in FIG. 10A, each of the locking hypotube features has a tapered proximal end 29a, which allows the sleeve to be slid over the hypotube, thereby pushing the inwardly extending resilient tabs of the sleeve outward, as shown in FIG. 10B. Further advancement of the sleeve allows the inwardly extending tabs to resiliently deflect inward to their set position and lock against a distal flat end 29b of the hypotube, as shown in FIG. 10C. The inwardly extending tabs 25a can be formed of any suitable material, including the same material as the collar or a differing material. In some embodiments, the one or more tabs are integrally formed with the collar. In other embodiments, the one or more tabs are separately formed and coupled with the collar. In some embodiments, the one or more tabs are formed of Nitinol and are set in the inwardly extended positions. As shown, the ring can lock onto any of the three locking hypotube features. This configuration allows the ring to accommodate variations in anchor positioning and depth relative the ring/annulus.

As shown in FIG. 11A, the anchor shaft 22 is attached to a locking collar 25 which includes a distally extending hook 26 that extends through a hole 27 in the anchor shaft 22 when the ring 10 and attached collar 25 is advanced over the torque wires 222, thereby locking the ring to the anchor. In another embodiment, the ring coupling mechanism includes a locking collar with a spring-loaded member that interfaces with a recess in the anchor body.

As shown in FIG. 11B, the locking collar 25 attached to the ring 10 includes a laterally extending, inwardly biased ball 28 that interfaces with the hole or detent 23. As shown in the detail view, member 28 includes a spring 28a that biases a distal ball 28b inwardly so that when the collar is advanced over the anchor, the ball 28b is forced by spring 28a into detent 23, thereby locking the ring to the anchor, after which the torque wire can be detached as described above. While these examples are shown with the cam lock couple-release mechanism, it is appreciated that these ring coupling mechanisms could be used with various other embodiments as well.

In some embodiments, the couple-release mechanism can be configured such that engagement the ring locking mechanism actuates the torque wire couple-release mechanism to decouple the torque wire. For example, engagement of inwardly biased ridge 23a with the anchor shaft 22 can actuate a member that decouples coupling features 24a,24b to allow release of the torque wire. This design is advantageous as locking of the ring with the lock mechanism effects release of the torque wires. While a particular design of the lock mechanism and couple-release mechanism are shown and described above, it is appreciated that these mechanisms can include any interfacing components or any suitable connectors configured to provide the functionality noted above.

In this embodiment, the anchor tip and shaft are fabricated from stainless steel, although any suitable material can be used. The anchor can be formed of an integral component or can include multiple components attached together. Typically, the anchors are provided as described with the lock mechanism and couple-release mechanism attached thereto. While screw anchors are described herein, it is appreciated that any suitable type of anchor can be used including barbed anchors that are driven into tissue by applying an axial force from driving members connected to the anchor shaft. In this approach, the anchors can be deployed and removed in a similar manner, selecting any, all or any combination of anchors.

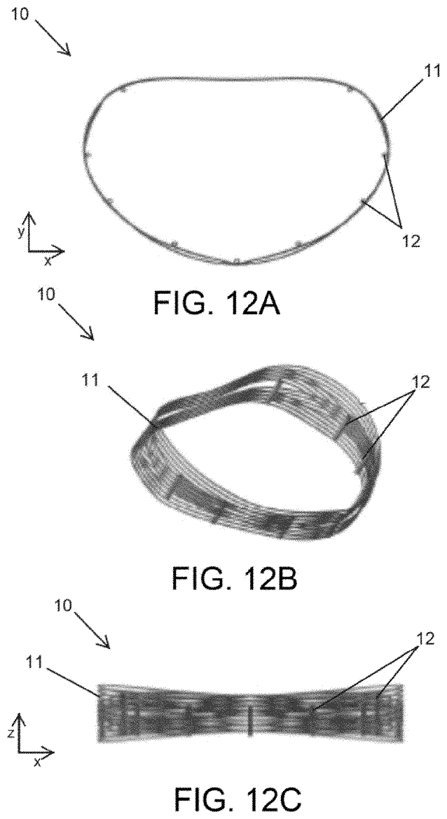

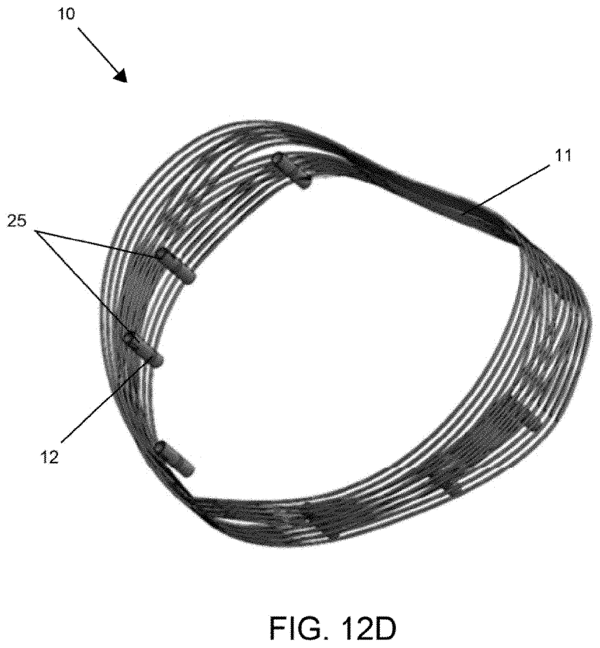

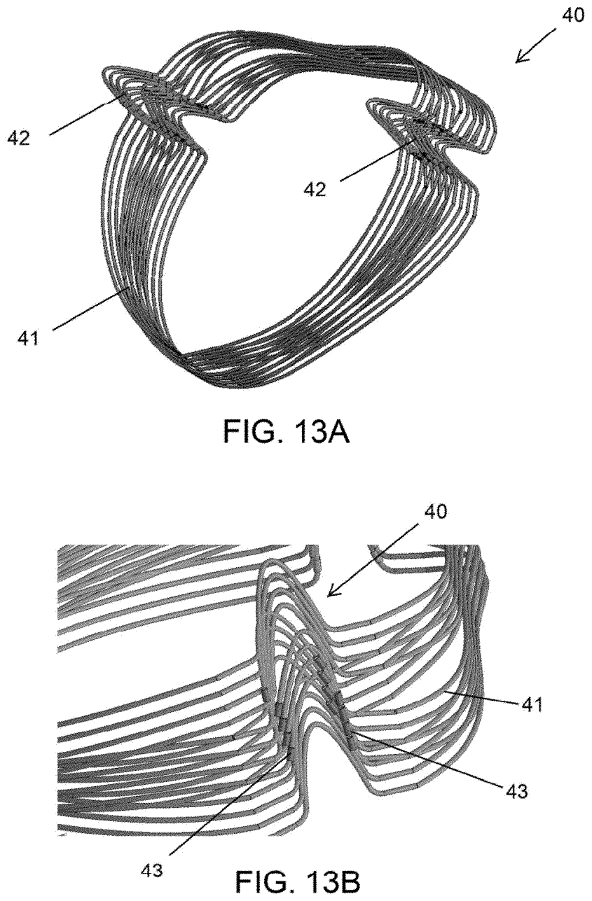

FIGS. 12A-12C show several views of an annuloplasty ring 10 in accordance with some embodiments. The ring 10 includes multiple concentric loops or rings 11 and a series of openings or eyelets 12 that receive the anchors to implant and secure the ring 11 against the valve annulus. In this embodiment, the annuloplasty ring is formed of a shape-memory alloy, such as Nitinol, and heat-set into three dimensional shape that mimics the healthy anatomical shape of the annulus. This allows the ring to be collapsed into a relatively small sized delivery catheter and to resume the desired shape when deployed from the catheter and secured to the anchors surrounding the valve annulus. Typically, the annuloplasty ring is semi-rigid. Advantageously, the three-dimensional design allows a variety of shapes and sizes to match the patient anatomy and specific characteristics of the mitral regurgitation in the patient, thereby providing a customized treatment approach. Evaluation of the patient pre-procedure with standard imaging techniques can be used to determine the shape and size ring for a given patient's anatomy. As shown in FIG. 12D, the ring 10 can include eyelets, each having a collar 25 to facilitate advancement of the ring over wires or cables. In this embodiment, the ring 10 includes eight collars at the eyelet locations, which are spaced non-uniformly at locations desired to anchor the ring along the valve. It is appreciated that the ring can include more or fewer collars at various other locations. The collar 25 can further include a ring locking feature, such as any of those described herein. In another aspect, the annuloplasty ring can be adjustable, for example as show in FIGS. 13A-13B described further below.

As shown, the annuloplasty ring 10 includes multiple concentric loops or rings that together form the ring structure. In some embodiments, the ring include any suitable number of loops, for example between 2 and 50, 5 and 30, or 10 and 20. The loops are generally of a similar 2D shape as each other, as can be seen in FIG. 6A, that corresponds to the desired 2D shape of the valve annulus. In this regard, the ring is similar to a shape of a conventional annuloplasty ring along two dimensions (x-y direction). However, the multiple loops can have differing shapes along the third dimension (z-direction), as can be seen from the side view in FIG. 6C. This 3D shape allows the annuloplasty ring to reform the valve annulus along an additional dimension, thereby better reforming the dilated valve annulus to a desired 3D shape to further improve coaptation of the leaflets of the valve. In one aspect, the annuloplasty ring designs can be optimized and evaluated for radial strength, ability to deploy and low profile.

In another aspect, the annuloplasty ring can include adjustable sections or portions that can be tightened or loosened to adjust the overall shape and/or size of the ring from outside the patient during deployment. In some embodiments, the function of the heart can be monitored during deployment and the ring adjusted accordingly until a desired heart valve function is achieved. In some embodiments, the ring includes v-shaped elements at specific locations that can be cinched tighter, as needed in order to reduce the size of the ring. As shown in FIGS. 13A-13B, the adjustable annuloplasty ring 40 includes multiple concentric wire loops 41 with two v-shaped elements 42. In the embodiment shown, the v-shaped elements 42 are located on opposite sides, along to major axis of the oval. This results in a reduction of the minor axis which corresponds to the septal-lateral direction on the valve, which is typically the most effective direction for mitral valve reduction. It is appreciated, however, that the adjustment portions could be located at various other locations and utilize various other constructions.

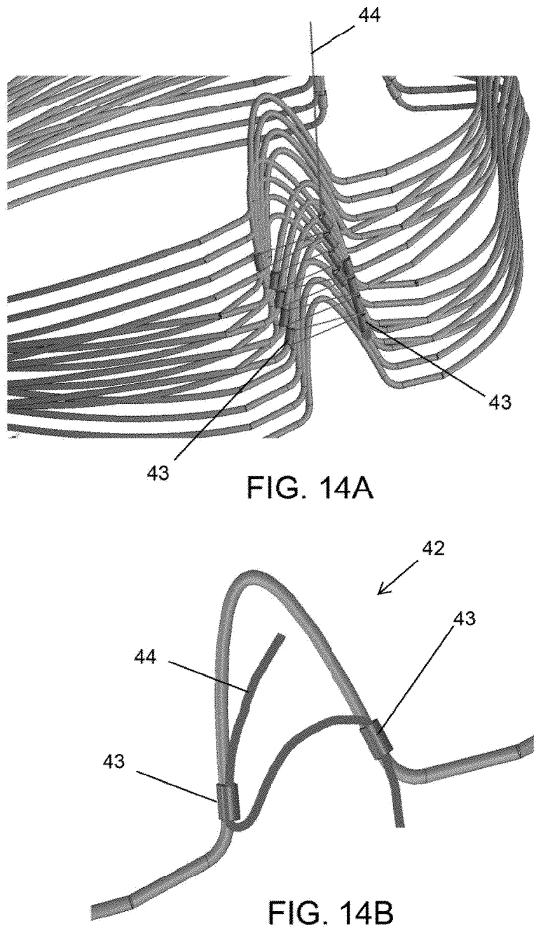

As shown in FIG. 13B, each wire of the v-shaped element includes a collar 43 on opposite sides. Collars 43 are fixed on the wider portions of the v-shaped element and designed so that a cable can be passed through the collars. As shown in FIGS. 14A-14B, cable 43 is positioned through the multiple collars so that it is fixed on one collar and routed to span each of the v-shaped elements and extends outside of the of the patient so that the v-shaped portion can be tensioned/tightened by the clinician during deployment of the implant system. When the cable 43 is tensioned, the collars are brought closer together, reducing the dimension along the v-shaped element.

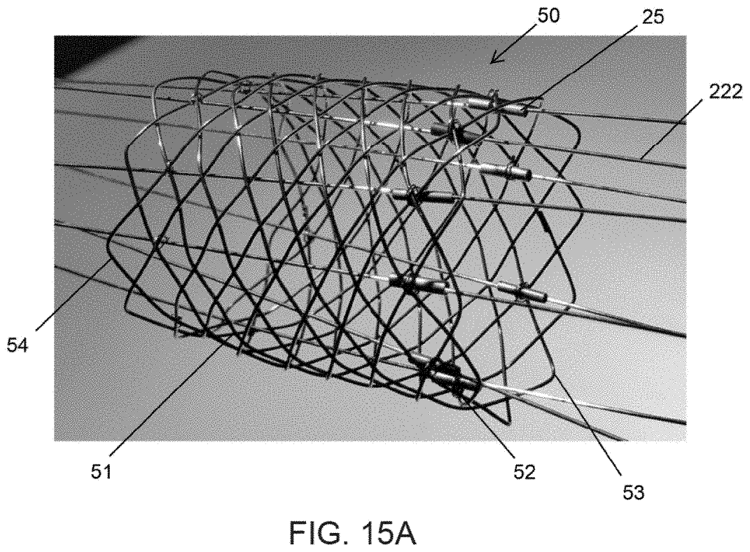

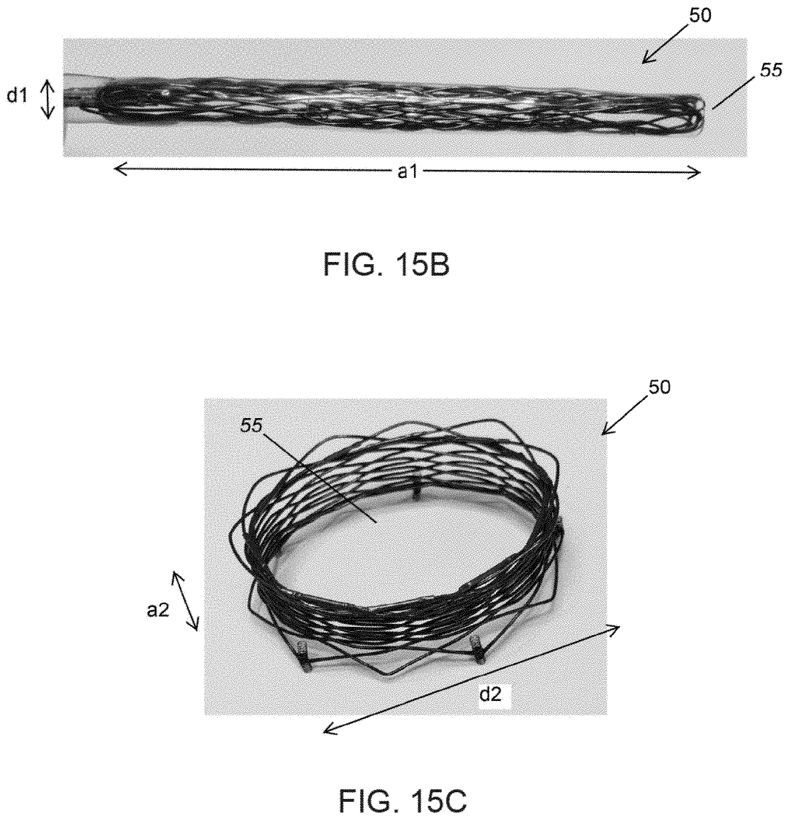

In another aspect, the annuloplasty ring can have a braided wire design that can be elongated and have a reduced diameter during delivery and then radially expanded to form the annuloplasty ring attached to the anchors. As shown in FIG. 15A, the annuloplasty ring 50 is designed as an expandable scaffold formed of braided wire 51 that is interwoven about a central opening. In this embodiment, the wire 51 is a shape memory alloy, such as Nitinol. The scaffold includes eyelets 52 disposed near a distal portion of the scaffold, the eyelets having a locking collar 25, as described previously. Preferably, the scaffold has top end 54 and bottom end 53 that are each atraumatic, for example, without any exposed wire ends. As shown, the wire ends are connected to each other within the braid to form a continuous wire braid. In this embodiment, the top and bottom ends have a zig-zag design with peaks and valleys. In FIG. 15A, the scaffold is shown being advanced along cable wires midway between the delivery configuration, shown in FIG. 15B, and the deployed configuration, shown in FIG. 15C.

In the delivery configuration shown in FIG. 15B, the scaffold is axially elongated such that axial dimension a1 is larger than the diameter d1. As shown, the axial dimension is about 10 times as long as the diameter such that the scaffold resembles an elongated tubular shape along the longitudinal axis. The first diameter is sufficiently small to fit through a vascular access sheath, preferably a 18 French access sheath or smaller to allow delivery of the implant system to the heart valve through the femoral artery. The first axial dimension is typically between 2 cm and 10 cm.

In the deployed configuration shown in FIG. 15C, the scaffold is radially expanded and axially collapsed such that the diameter d2 is greater than the axial dimension a2. As shown, the average diameter is about five times greater than the axial dimension. When formed of a shape memory alloy, such as Nitinol, the scaffold is heat set into this deployed implantation configuration such that once delivered into the heart, the scaffold assumes this configuration. As shown, the scaffold resembles an oval shaped ring extending circumferentially about the central opening 55. Typically, the diameter d2 is within a range of 2 cm to 4 cm and suited for being secured around a heart valve, such as the mitral valve. The axial dimension a2 is relatively small, typically within a range of 0.5 cm to 3 cm.

FIG. 15D shows an exemplary annuloplasty implant system 100 implanted on a model of a mitral valve annulus (MV) in accordance with some embodiments. In accordance with the embodiments described above, the implant system includes annuloplasty ring 50 and multiple screw anchors 20 implanted in tissue surrounding the MV. As can be seen, the torque wires 220 are still attached to the proximal end of the anchors 20 and the implant 50 has been advanced over the torque wires extending through the eyelets 12 and collars 25 and assumed the deployed configuration adjacent the annulus. The ring can then be locked to the anchor shafts while the torque wires 222 are decoupled from the anchors and removed leaving the implant in place. In some embodiments, the function of the valve can be assessed before the ring is locked into place so that adjustments can be made to the anchors or ring before decoupling the torque wires.

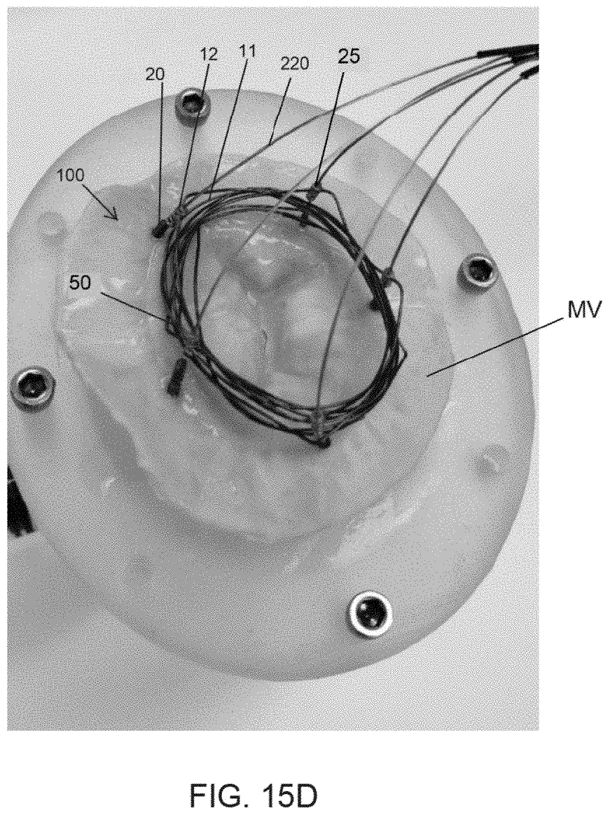

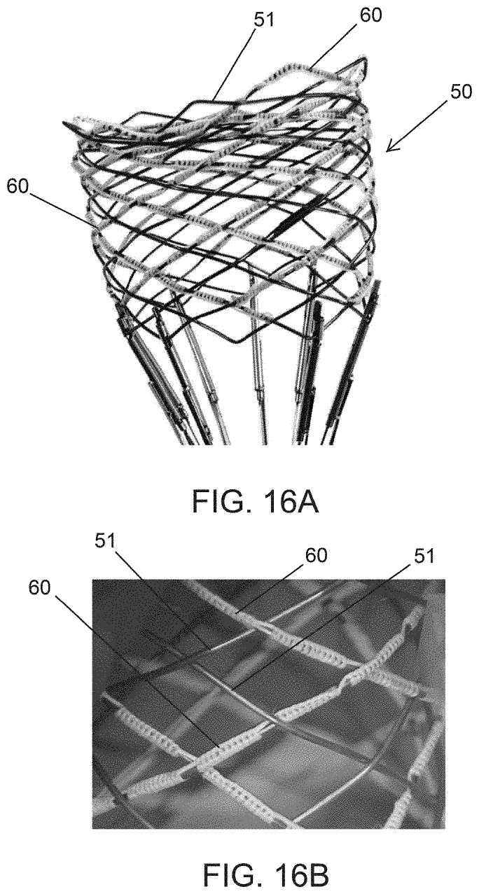

In some embodiments, the annuloplasty ring can include one or more tissue ingrowth features that promote tissue growth around implant to secure ring implant to the mitral annulus after implantation. These features can include but are not limited to coatings, sutures, filaments, biodegradable polymers, mesh or fabric disposed on select portions of the annuloplasty ring structure. FIGS. 16A-16B show an exemplary annuloplasty ring 50 comprised of braided wires 51 that include a tissue ingrowth feature of a braided polyester yarn or suture 60 that are wrapped about every other wire of the structure. In the embodiment shown, the ring is defined by loops of Nitinol wire. The suture 60 is wrapped and secured with a series of knots around the wires, avoiding wire crossover points to reduce fraying or damaging suture. By covering every other wire and avoiding wire crossover points, the suture does not restrict expansion of the implant. In some embodiments, other biocompatible fabrics, coatings or surface modifications can be added to the wires to improve tissue or blood interaction with the implant.

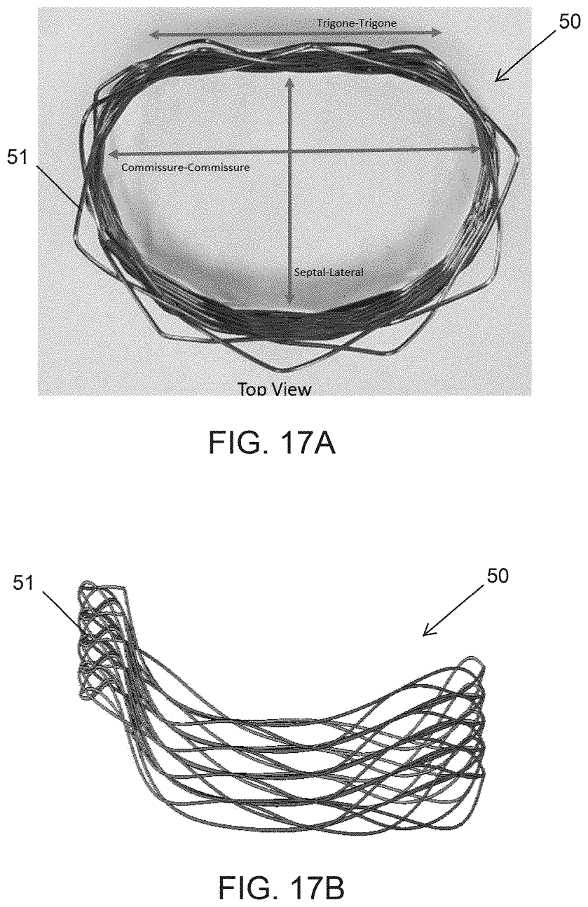

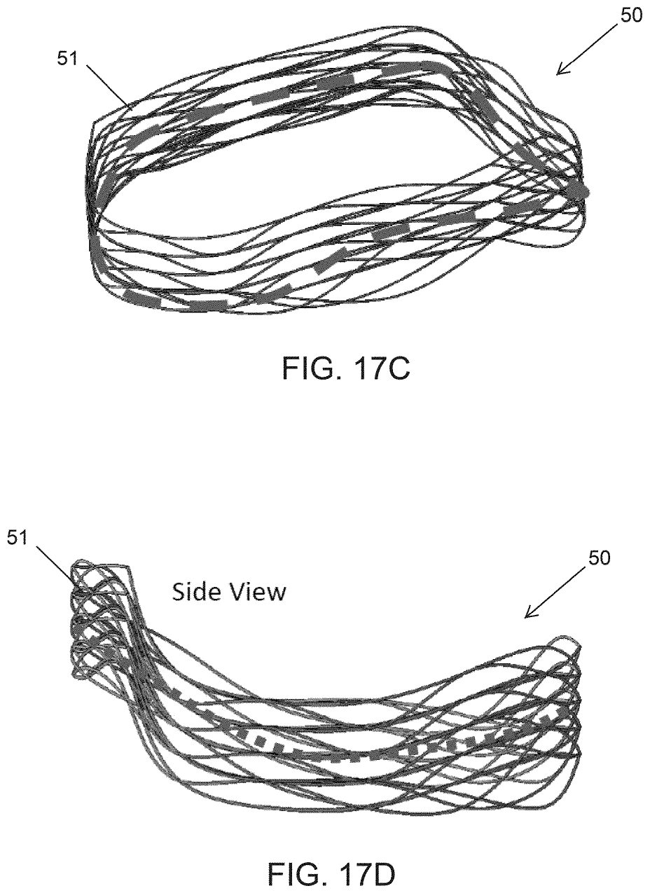

FIG. 17A shows an exemplary annuloplasty ring 50 that has been formed in a two-dimensional shape of D-shaped ring to better conform the annulus to a natural shape of a healthy mitral valve. Specifically, the D-shape has specific dimensions that correspond to relative to anatomic features within the mitral annulus, as shown in FIG. 1C, such that the ring is designed to reshape the heart in an anatomically advantageous shape, similar to a healthy annular shape. Rings can also be shaped to preferentially shape specific sections of the valve annulus depending on the patient. In some embodiments, the annuloplasty ring is further designed to assume a 3-dimensional shape that corresponds to a natural shape of a health mitral valve annulus, which resembles a saddle-shape, as can be seen in FIG. 1C. As shown in FIGS. 17B-17D, the multiple wire loops of the annuloplasty ring, which are typically Nitinol wire, can be formed/set along this desired shape (indicated by dashed line) and thereby provide a more anatomically correct remodeling of the heart.

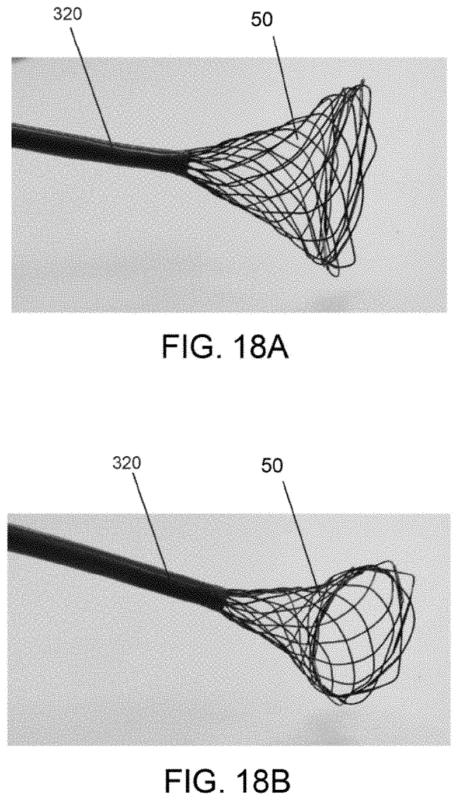

FIGS. 18A-18B shows the annuloplasty ring 50 being deployed from a ring deployment catheter. As can be seen, the annuloplasty ring can be constrained within a relatively small lumen of a catheter shaft 320 of the delivery catheter. The flexible braided scaffold design allows the entire ring to be axially elongated and radially collapsed and drawn into the catheter. The braided design has a mesh-like appearance, as shown in FIGS. 18A-18B, before the is distally advanced and deployed to form the annuloplasty ring.

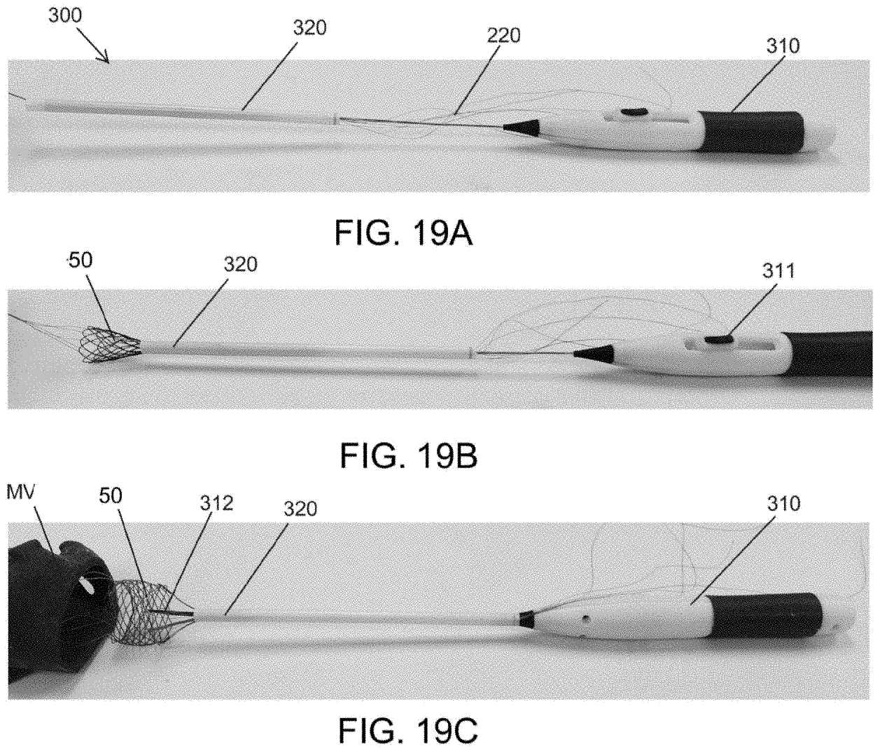

FIGS. 19A-19C show several views of an annuloplasty ring delivery catheter 300 in accordance with some embodiments. The delivery catheter 300 includes a proximal handle 310, an elongate flexible shaft 320, and an annuloplasty ring 50 constrained within a distal portion of the shaft. After removal of the anchor delivery catheter, the torque wires are left in place and the proximal ends of the torque wires are fed through the eyelets of the annuloplasty ring and then the ring is compressed and loaded into the shaft 320 with the torque wires 220 extending proximally from the shaft, as shown in FIG. 19A. The entire assembly is advanced over the torque wires to the mitral annulus. The ring can be deployed by proximal retraction of the shaft and/or by advancement of the pusher members 312 that engage the ring. The pusher members 312 extend to a control switch 311 on the handle. In this embodiment, the pusher elements are attached to the smaller catheter shaft which is attached to the handle. Advancement of the handle body will deploy the ring. Retraction of the handle body will pull the ring back into the larger shaft. The control switch on the handle disengages the pusher members from the ring and releases the ring from the catheter. Once released, the ring assumes its deployed configuration and can be attached to the anchors around the valve annulus, as described above.

As shown in FIG. 19C, pusher member 312 can include multiple arms that engage the ring to facilitate advancement and deployment of the ring adjacent the valve annulus. At this point, the shape and/or function of the reformed valve can be assessed by visualization techniques. If the physician determines the shape of the valve or valve performance is unsatisfactory, the ring can be removed by pulling the torque wires taut from the proximal end and drawing the ring within the sheath. The ring can then be withdrawn and adjusted or replaced as needed and the procedure repeated and re-assessed. Once the shape of the valve and/or valve function is satisfactory, the ring can be further advanced to secure the ring to the lock mechanism of the anchor shafts by the ring locking mechanism and decouple the torque wires from the anchors by the couple-release mechanism.

As shown, the pusher element comprises multiple arms that splay laterally outward and engage the most proximal loop of the prosthetic to allow axial movement of the pusher member to advance or retract the ring. The arms can be engaged with the loop by hooks, a coupling mechanism or any suitable releasable connector. In some embodiments, the pusher member can include one or more tubes disposed over one or more of the torque wires. While the ring delivery catheter is described as a separate catheter that is used after removal of the anchor delivery catheter, it is appreciated that the catheters can be combined within a single catheter in some embodiments.

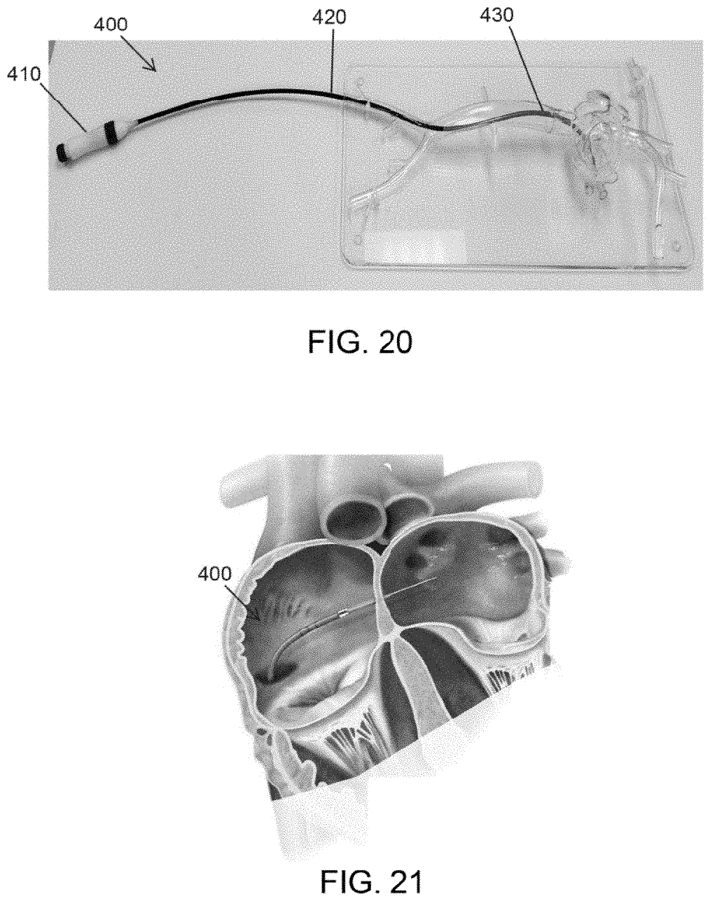

FIG. 20 shows an articulable access sheath 400 that can be advanced intravascularly to an atrium of the heart to provide access for the respective delivery catheters of the anchors and annuloplasty ring in accordance with some embodiments. The access sheath can include a proximal handle 410 with proximal access opening, an elongate flexible sheath body 420 and a flexible articulable distal region 430. In some embodiments, the access sheath is a deflectable 20F sheath to aid in delivery and positioning of the implant system. This access sheath allows the above-noted implantation procedure to be performed in a transfemoral-transseptal approach from a venous access site. The mitral valve can be accessed from the atrial side by a right to left atrial puncture. FIG. 21 shows the access sheath advanced through the septal wall and into the left atrium to provide access to mitral valve in the left atrium.

FIGS. 22A-22H show sequential views of an exemplary method of delivery and implantation of the annuloplasty implant system in accordance with some embodiments.

In FIG. 22A, the delivery catheter is advanced to the mitral valve from the atrial side. The assembly of the anchor support 230 and centering member 240 is then advanced so that the center shaft 241 of the centering basket enters the mitral valve, as shown in FIG. 22B. As shown, the assembly is positioned so that the center shaft of the centering assembly extends through the valve annulus into the ventricle, while the anchor support frame remains above the valve annulus in the atrium. The position of the assembly within the valve annulus can be confirmed by visualization techniques.

As shown in FIG. 22C, the centering member 240 is expanded within the valve annulus (for example by axial movement of a control switch on the proximal handle), thereby centering the assembly within the valve annulus. As can be seen, since the anchors 20 are supported further outside of the centering member, thereby positioning anchors surrounding the valve annulus. If needed, the anchor support 230 can be further advanced to ensure sufficient contact with surrounding tissues. As discussed previously, the anchor support can include spring portions that allow the anchors more leeway and conformability so that all anchors can suitably engage with surrounding tissue regardless of uneven contours of the tissues. Advantageously, the centering member can be a basket or scaffold to allow blood flow between the atrium and the ventricle even during the centering procedure.

As can be seen in FIG. 22D, the centering member has been contracted and axially retracted into the delivery catheter. Advantageously, this allows the valve to function while the physician continues the process of securing the anchors into the surrounding tissue. While the anchor support 230 supports the torque wires (not shown) and anchors in the proper position, the physician actuates the torque wires to drive the screw anchors into the surrounding tissue. As noted above, the physician can select any, all, or any combination of the screw anchors or can explant individual anchors as needed. Preferably, multiple anchors are deployed concurrently, which improves the ease of implantation and reduces the length of the overall procedure.

As shown in FIG. 22E, after the screw anchors 20 are satisfactorily implanted in the surrounding tissue, the anchor support can be withdrawn, along with the delivery catheter, leaving the torque wires in place extending through access sheath 400. The annuloplasty ring is then fed onto the proximal ends of the torque wires via the eyelets and loaded into the ring delivery catheter as described previously.

As shown in FIG. 12F, the annuloplasty ring is then advanced from the ring delivery catheter 300 over the torque wires 221). As can be seen in FIG. 12G, the ring can be further advanced from the catheter by a pusher member(s) 312 so that the scaffold emerges from the delivery sheath and assumes the deployed configuration and then is secured to the anchors adjacent the valve annulus. At this point, the shape of the reformed valve and/or valve function can be assessed, and if needed, the ring can be retracted and adjusted or replaced based on the assessment. Once the physician determined the shape of the reformed valve and/or valve function is suitable, the annuloplasty ring 10 is locked to the anchor shaft via a lock mechanism (for example, by further advancement of the ring) and the torque wires are decoupled from the anchor shafts. The ring delivery catheter and access sheath can then be removed, leaving the annuloplasty implant system in place, as shown in FIG. 22H.

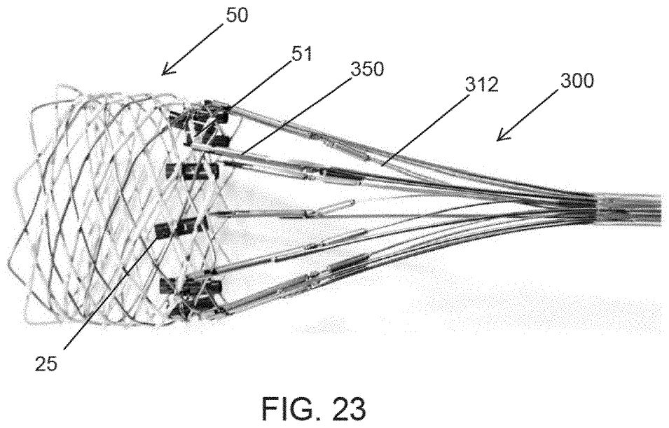

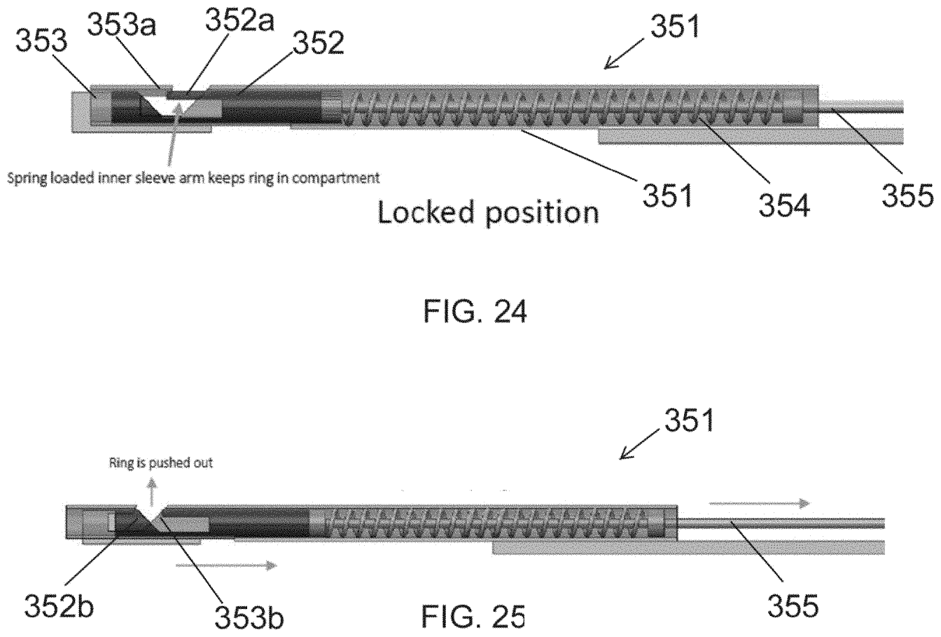

In the embodiment of FIG. 23, the implant delivery catheter 300 includes pusher members 312 that each include an implant holding-release mechanism 350 on a distal portion thereof, which releasably engages the ring 50 while it is being advanced over the cables, and release the ring after locking of the collars 25 to the anchors. The ring holding-release mechanism 350 can be further understood by referring to FIGS. 24 and 25 which depict the mechanism in the locked and released positions, respectively.

As shown in FIGS. 24 and 25, each ring holding-release mechanism 350 is a spring loaded hypotube sleeve 351, which includes an inner retractable hypotube sleeve 352 that retains ring 50 in the locked position constrained between inner sleeve 352 and outer hypotube sleeve 353 when the inner sleeve is pushed in the fully extended position by spring 354. As shown, each hypotube sleeve has teeth 352a, 353a that engage a wire of the ring in the locked position. The spring loaded release mechanism can be released from the proximal end of the catheter by retracting pull wire 355, which is attached to and retracts the inner sleeve and compresses the spring. Additional spring length creates slack and prevents inadvertent pre-release of any individual prong arm during delivery. While the ring is locked to the mechanism 350, the ring 50 is advanced over the torque wires and against the anchors, thereby locking the collars of the ring to the anchors, as described previously. Once the ring is locked in place, the pull wire(s) can be pulled to release the spring-mechanism and retract the inner sleeve(s) 352 proximally, thereby releasing the ring 50 from all the pusher members. In this embodiment, a wedge surface 352b, 353b on both the inner and outer hypotube sleeves interface when in the released position, thereby forcing the ring out when the inner sleeve retracts. In some embodiments, the pushing members can be locked together at the proximal end and pushed together so as to maintain planarity and uniform advancement of the ring along the cables. In some embodiments, push members can be individually controlled or advanced further relative other pushing members so as to conform the ring to a non-planar shape against the annulus. After release, the pusher members can then be retracted into the outer sheath of the implant delivery catheter and withdrawn from the body.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art. Each of the references cited herein are incorporated herein by reference for all purposes.

1. An annuloplasty ring comprising:
a scaffold formed by one or more wires that are braided circumferentially about a central opening extending along a longitudinal axis of the scaffold, wherein the scaffold is expandable from a delivery configuration to a deployed implantation configuration;
wherein in the delivery configuration, the scaffold has first axial dimension and a first diameter about the central opening, the first axial dimension being greater than the first diameter; and
wherein in the deployed implantation configuration, the scaffold has a second axial dimension and a second diameter about the central opening, wherein the second diameter is greater than the second axial dimension.

2. The annuloplasty ring of claim 1, further comprising:
a plurality of eyelets disposed circumferentially about the scaffold and configured to allows passage of wires therethrough to facilitate delivery of the scaffold over a plurality of wires.

3. The annuloplasty ring of claim 1, further comprising:
a plurality of collars distributed circumferentially about the scaffold and configured to allows passage of wires therethrough to facilitate delivery of the scaffold over a plurality of wires.

4. The annuloplasty ring of claim 3, wherein each of the plurality of collars includes a plurality of inwardly extending tabs that are inclined in a proximal direction so as to facilitate passage over a lock of an anchor shaft coupled to a respective wire of the plurality of wires and to facilitate locking with the respective anchor by abutting against a distal facing surface of the lock of the anchor shaft.

5. The annuloplasty ring of claim 1, wherein the scaffold is radially expandable and axially collapsible to allow the scaffold to deploy from the delivery configuration to the implantation configuration when advanced along the plurality of wires.

6. The annuloplasty ring of claim 1, wherein the delivery configuration is an elongate tubular shape along the longitudinal axis.

7. The annuloplasty ring of claim 1, wherein the deployed configuration is a ring.

8. The annuloplasty ring of claim 1, wherein the first diameter is sufficiently small to fit through a vascular access sheath.

9. The annuloplasty ring of claim 1, wherein the first diameter is sufficiently small to fit through a 7 French access sheath or smaller.

10. The annuloplasty ring of claim 1, wherein the first axial dimension is between 2 cm and 10 cm.

11. The annuloplasty ring of claim 1, wherein the second diameter is within a range of 2 cm to 6 cm.

12. The annuloplasty ring of claim 1, wherein the second axial dimension is within a range of about 0.5 cm to 3 cm.

13. The annuloplasty ring of claim 1, wherein the scaffold is formed of Nitinol wire.

14. The annuloplasty ring of claim 1, wherein the scaffold has a design with atraumatic proximal and distal ends about the central opening.

15. The annuloplasty ring of claim 14, wherein the proximal and distal ends comprise a zig-zag design having a plurality of peaks and valleys.

16. The annuloplasty ring of claim 1, further comprising a plurality of eyelets distributed at or near the bottom end nearest the valve annulus when implanted.

17. The annuloplasty ring of claim 1, further comprising a plurality of collars at the plurality of eyelets to facilitate sliding of the ring along the plurality of wires.

18. The annuloplasty ring of claim 1, wherein the plurality of collars include a ring locking feature that facilitates coupling of the scaffold to a plurality of anchors disposed around a heart valve.

19. The annuloplasty ring of claim 18, wherein the ring locking feature includes one or more inwardly extending tabs inclined in a proximal direction.

20. The annuloplasty ring of claim 18, wherein the ring locking feature includes a protruding member of the collar that is received within an opening or recess in the anchor.

21. The annuloplasty ring of claim 18, wherein the ring locking feature comprises a resilient ridge along an inner facing surface of the collar that is biased inwardly in a proximal direction to allow the resilient ridge to be advanced over a proximal shoulder in the anchor shaft and deflect inward so that the ridge abuts against the shoulder thereby locking the ring to the anchor.

22. The annuloplasty ring of claim 18, wherein the ring locking feature comprises a curved hook that deflects toward the anchor shaft so that when advanced over the anchor, the curved hook protrudes within a hole or recess within the anchor shaft thereby locking the ring to the anchor.

23. The annuloplasty ring of claim 21, wherein the ring locking feature comprises a spring-loaded ball or peg that is biased toward the anchor shaft so that when advanced over the anchor, the spring-loaded ball or peg protrudes into a hole or recess within the anchor shaft thereby locking the ring to the anchor.

24. The annuloplasty ring of claim 1, further including a polymer suture wrapped around select portions of the one or more wires so as to promote tissue ingrowth after implantation to further secure the implant.

25. The annuloplasty ring of claim 24, wherein the polymer suture is wrapped around at least some of the concentric loops and avoids cross-over areas of adjacent loops to allow expansion of the scaffold.

26. The annuloplasty ring of claim 1, wherein the one or more wires are Nitinol wire that is heat set to the deployed configuration in which the concentric wire loops define a D-shape.

27. The annuloplasty ring of claim 26, wherein one or more wires are further heat set to assume a three-dimensional saddle shape.

28. An annuloplasty ring comprising:
a plurality of concentric rings flexibly interconnected by a plurality of struts such that the plurality of rings are separable axially,
wherein the plurality of concentric rings are formed of a shape memory alloy that is heat set to an implantation configuration that corresponds to desired characteristics of a native heart valve,
wherein the plurality of rings are deformable into a contracted configuration for delivery through a catheter and return to the implantation configuration when released from the catheter during implantation; and
a plurality of eyelets or collars distributed circumferentially about the plurality of concentric rings for interfacing with a plurality of anchors or anchor wires.

29. The annuloplasty ring of claim 28, wherein the shape memory alloy is Nitinol.

30. The annuloplasty ring of claim 28, wherein the plurality of concentric rings have substantially similar 2-dimensional shapes within a plane along which the ring is defined.

31. The annuloplasty ring of claim 30, wherein the 2-dimensional shape corresponds to a desired shape of the valve annulus.

32. The annuloplasty ring of claim 31, wherein the 2-dimensional shape is a D-shape.

33. The annuloplasty ring of claim 30, wherein the plurality of concentric rings have differing 3-dimensional shapes.

34. The annuloplasty ring of claim 33, wherein together the rings define a 3-dimensional shape that is a saddle-shaped.

35. The annuloplasty ring of claim 33, wherein the differing 3-dimensional shapes are designed such that, when combined, the annuloplasty ring has a radial strength and flexibility corresponding to the desired characteristic of the native valve annulus.

36. The annuloplasty ring of claim 35, wherein the plurality of rings are customized to provide a shape, strength and flexibility for the valve annulus of a particular patient.

37. The annuloplasty ring of claim 28, wherein the eyelets or collars are dimensioned to receive a plurality of anchor shafts and anchor wires attached thereto.

38. The annuloplasty ring of claim 28, wherein the plurality of rings and struts are connected such that the plurality of rings are separated in an axial direction in the contracted configuration and the rings are collapsed together in axial direction to form the annuloplasty ring in the implantation configuration.

39. The annuloplasty ring of claim 28, wherein the plurality of rings and struts form a mesh-like pattern.

40.-75. (canceled)
